(19) United States
(12) Patent Application Publication
(10) Pub. No.: US 2011/0276090 A1
(43) Pub. Date: Nov. 10, 2011

(54) MEDICAL PRODUCT , A SURGICAL KIT, AND A METHOD FOR PRODUCING THE MEDICAL PRODUCT

(75) Inventors: Ingo Berndt, Aachen (DE); Erich Odermatt, Schaffhausen (CH); Erhard Müller, Stuttgart (DE); Sven Oberhoffner, Weinstadt-Endersbach (DE)

(73) Assignees: ITV Denkendorf Produktservice GmbH, Denkendorf (DE); Aesculap AG, Tuttlingen/Donau (DE)

(21) Appl. No.: 13/102,424

(22) Filed: May 6, 2011

Related U.S. Application Data
(63) Continuation of application No. PCT/EP2009/007949, filed on Nov. 6, 2009.

(30) Foreign Application Priority Data
Nov. 6, 2008 (DE) 10 2008 057 213.6

(51) Int. Cl. A61B 17/06; B23K 26/00; A61L 17/12; B26D 3/00; A61L 17/04; A61L 17/06

(52) U.S. Cl. 606/230; 606/228; 606/222; 606/231; 83/870; 219/121.69

(57) ABSTRACT

A medical product including a biocompatible material includes two mutually opposite main surfaces, wherein at least one of the two main surfaces that anchor biological tissues, wherein the barbs protrude from the at least one main surface and are formed by cuts made in the biocompatible material, and wherein the material between the two main surfaces is completely broken through in an area of the cuts.

### RELATED APPLICATIONS

This is a continuation under §111 of International Application No. PCT/EP2009/007949, with an international filing date of Nov. 6, 2009 (WO 2010/052006 A1, published May 14, 2010), which is based on German Patent Application No. 10 2008 057 213.6, filed Nov. 6, 2008, the subject matter of which is incorporated by reference.

### TECHNICAL FIELD

This disclosure relates to a medical product, a surgical kit, and a method for producing the medical product.

### BACKGROUND

Thread-like suture materials are used as standard in surgery for closure of wounds. They are usually knotted to ensure a secure anchoring in the tissues that are to be closed. Care has to be taken to ensure that the wounds to be closed are always sutured with an optimal force at the wound margins. If the wound margins are sutured too loosely and too irregularly, there is in principle a risk of increased scar formation or dehiscence. By contrast, if the wound margins are sutured too strongly, there is a danger of the circulation of blood in the wound margins being restricted, which can result in necrotic changes in the surrounding tissue area. Apart from the possible complications that can arise as a result of this and that may in some cases necessitate further surgical interventions, there is also a risk that the closure techniques using knots may provide unsatisfactory cosmetic results in the patients concerned. A further consideration is that several knots, in particular up to seven knots, often have to overlap to achieve a secure knot hold. This entails introducing a large amount of material into the tissue that is to be closed and can lead to increased foreign-body reactions.

Suture materials that are knotless or self-fixing, so-called “self-locking” sutures, therefore promise a number of advantages. Thus, the omission of knots ought in general to lead to more rapid wound repair, in particular wound closure. In addition, there ought to be less risk of wound dehiscence when using knotless suture materials. By omitting knots, less foreign material would also be introduced into the tissue that is to be treated. Moreover, as regards scar formation too, better results can be expected when using knotless suture materials, which is of special importance particularly in plastic or cosmetic surgery.

Knotless or self-fixing suture materials have long been known of in the form of barbed sutures. These usually comprise a monofilament thread which has barb-like structures, of barbs, along its longitudinal axis. Corresponding suture materials have already been described in U.S. Pat. No. 3,123,077 A. Moreover, barb-like suture materials are described, for example, in EP 1 559 266 B1, EP 1 560 683 B1 and EP 1 555 946 B1. The barbs are usually produced by making cuts in the thread material. In this way, the threads can be pulled through the tissue along the direction of the barbs, without any great resistance and without causing any great trauma. When a pull is exerted in the opposite direction, the barbs stand upright and anchor themselves, and therefore the suture material, in the tissue. This ensures that the suture material cannot be pulled back through the incision channel.

It could therefore be helpful to make available a medical product which in particular, compared to conventional knotless suture materials, permits an improved hold in biological tissues. Moreover, such a product should be as easy as possible to produce.

### SUMMARY

We provide a medical product including a biocompatible material including two mutually opposite main surfaces, wherein at least one of the two main surfaces has barbs that anchor biological tissues, wherein the barbs protrude from the at least one main surface and are formed by cuts made in the biocompatible material, and wherein the material between the two main surfaces is completely broken through in an area of the cuts.

We also provide a surgical kit including the medical product and at least one surgical needle.

We further provide a method for producing the medical product, wherein a preliminary stage of the medical product, which is made of a biocompatible material and has two mutually opposite main surfaces, is cut to form barbs, with the material of the preliminary stage being broken through completely between the two main surfaces, and the barbs are then converted to a position in which they protrude relative to at least one of the two main surfaces.

### BRIEF DESCRIPTION OF THE DRAWINGS

FIG. 1 shows an example of our product.

FIG. 2 shows a plan view of an example of the product.

FIG. 3 shows a plan view of another example of the product.

FIG. 4 shows a side view of a further example of the product.

FIG. 5 shows another example of the product.

FIG. 6 shows yet another example of the product.

FIG. 7 shows a further example of the product.

FIG. 8 shows still another example of the product.

### DETAILED DESCRIPTION

Our medical product is made of a biocompatible material comprising two mutually opposite main surfaces, wherein at least one of the two main surfaces has barbs for anchoring in biological tissues, particularly human and/or animal tissues, wherein the barbs protrude from the at least one main surface and are formed by cuts made in the biocompatible material, wherein the material between the two main surfaces is completely broken through in the area of the cuts.

Main surfaces are to be understood as the surfaces of the medical product that have the greatest extent.

The at least one main surface, in particular both main surfaces, can in principle have different contours. For example, the contours can be circular or polygonal. In particular, the at least one main surface, in particular both main surfaces, can have rounded corners. Both main surfaces preferably each have a square, in particular rectangular, contour.

Preferably, the barbs are formed in the longitudinal direction of the at least one main surface. The barbs on the at least one main surface can have a unidirectional or bidirectional arrangement or configuration. A bidirectional arrangement is particularly preferred. A bidirectional arrangement is to be understood as an arrangement of barbs in which the barbs on the at least one main surface are oriented in two different directions. Seen in the longitudinal direction of the medical product, the barbs for a first surface portion of the at least one main surface are preferably formed in the direction of another, second surface portion of the at least one main surface and, for the other, second surface portion, are formed in the direction of the first surface portion.

It is particularly preferable if, as seen in the longitudinal direction of the medical product, the barbs for a first surface portion of the at least one main surface are formed in the direction of the center of the at least one main surface and, for another, second surface portion of the at least one main surface, are likewise formed in the direction of the center of the at least one main surface. The length of the surface portions particularly preferably corresponds to approximately half of the at least one main surface, seen in the longitudinal direction of the at least one main surface. In this way, the medical product can be pulled from one end thereof to approximately its center through a biological tissue, without any great resistance and without causing any trauma that is relevant from a medical point of view and, when a pull is exerted in the opposite direction, the barbs stand upright and in this way anchor or fix the medical product in the tissue, without knots being needed.

The barbs on the at least one main surface can in principle be formed in different arrangements. Thus, the barbs in the longitudinal and/or transverse direction, preferably in the longitudinal direction, of the at least one main surface can have a row arrangement, an offset arrangement, a zigzag arrangement, an overlapping arrangement, an offset and partially overlapping arrangement, a meandering arrangement, a serpentine-type arrangement, a sinus-shaped arrangement, an arbitrary arrangement, or combinations of these.

The barbs may have an offset arrangement, wherein the barbs are partially overlapping one another. Such an arrangement may be realized, for example, by forming barbs with a small angular offset and in small intervals between each other on the medical product, preferably by shallow cuts into the medical product. In such an arrangement, two adjacent barbs form a barb having a twin-tip configuration (“double-acting” barb). Such a twin-tip configuration is primarily advantageous in relation to solid and secure anchoring of the medical product in biological tissue.

The at least one main surface may have at least two sets, preferably two identical sets, of barbs. A set of barbs is to be understood here as meaning ah arrangement of barbs which corresponds in respect of the configuration of the barbs, in particular in respect of the height of the barbs, the length of the barbs, the angle of cut of the barbs, the orientation of the barbs and/or the shape of the barbs. The at least one main surface preferably has at least two, in particular two, sets of barbs, which are arranged parallel to each other on the at least one main surface. In this way, the number of barbs per unit of length can be particularly advantageously increased on the product as a result of which the anchoring ability of the product in biological tissues is improved.

The barbs themselves can in principle be designed in different geometries. For example, the barbs can be scale-shaped, escutcheon-shaped, shield-shaped, V-shaped, W-shaped, wedge-shaped, thorn-shaped and/or arrow-shaped. The barbs are preferably pointed or tapered at their end protruding from the at least one main surface.

The barbs may protrude from the at least one main surface at an angle α of between 0 and 90 degrees, in particular 15 and 75 degrees. Moreover, the barbs, particularly barbs formed in the longitudinal direction of the at least one main surface, can have a mutual spacing of 0.1 to 5 mm, in particular 0.2 to 3 mm. A mutual spacing of the barbs of <2 mm is particularly preferred.

Advantageously, the barbs are formed on both main surfaces of the medical product. It is particularly preferable if the barbs are offset relative to one another on the mutually opposite main surfaces. The examples described in this paragraph have the advantage that the barbs provide anchoring possibilities on two sides of the medical product, which contributes to further optimize the anchoring and fixing of the product in biological tissues. For further details and features, particularly regarding the possible arrangements arid configurations, reference is made in full to the preceding description.

Particularly advantageously, barbs are additionally formed on at least one side surface, particularly on two side surfaces, preferably on two mutually opposite side surfaces, of the medical product. In other words, the medical product can have laterally formed barbs. It is particularly preferable if the at least one side surface or the two side surfaces is/are at least one longitudinal side surface or two longitudinal side surfaces of the medical product. In this way, the number of the barbs can be increased, and thus the hold of the medical product in biological tissues enhanced, in a particularly effective way. The laterally formed barbs can in principle be present in all possible arrangements and configurations. Reference is made in this connection to the preceding description.

The barbs may be formed by cuts made in the medical product when the latter is in a drawn state. Alternatively, the barbs can also be formed by cuts made in the medical product when the latter is in an undrawn state. The barbs can particularly advantageously be made to stand upright by subsequent drawing in the longitudinal direction of the medical product.

In areas without barbs, the medical product preferably has a square, in particular rectangular, cross section. It is also preferable that the medical product is flat. The product particularly preferably has a greater width than thickness. The medical product can have a width-to-thickness ratio of between 100 and 1, particularly 50 and 2, preferably 20 and 8. Thus, the medical product can have a width of between 0.1 and 5.0 mm, in particular 0.2 and 3 mm, preferably 0.5 and 2 mm. In addition, the medical product can have a thickness of between 0.05 and 5 mm, in particular 0.06 and 1.5 mm, preferably 0.1 and 0.25 mm.

The medical product may be strip-shaped or band-shaped. The medical product is preferably in the form of a band. The medical product is particularly preferably provided as a film, in particular as a film tape. The medical product is also provided as an extrusion product, in particular as an extrusion film.

Advantageously, the medical product, at least at one end, has an extension, in particular a web-shaped extension, for fitting with a surgical needle, wherein the extension preferably has a width corresponding to the thickness of the medical product. The extension is generally made of the same material as the medical product and is preferably connected in one piece to the latter. The extension preferably has a length corresponding to the length of a drilled hole in the shaft of a surgical needle.

The medical product may have an opening, in particular a slit-shaped opening, at one end. The opening can be formed transversely, obliquely or longitudinally in the one end. An opening transverse to the longitudinal direction of the medical product is preferred. The opening can also be formed as a loop at one end of the medical product. The opening is provided to allow the medical product to be pulled through from its other end, particularly in the manner of a cable tie. The barbs of the medical product advantageously prevent the medical product from sliding back out of the opening. In practice, the medical product can in this way be guided through the slit after a first passage through tissue. As has already been mentioned, the barbs prevent the product from sliding back, resulting in a first knotless fixing of a continuous suture. The area with the slit-shaped opening can be wider than the other areas of the medical product.

The medical product or the main surfaces of the medical product may be profiled, preferably formed in a zigzag or curved structure. The medical product or its main surfaces may be provided in a folded structure, for example. The barbs are preferably formed on or along profile elevations or projections of the product or its main surfaces. Preferred is that the barbs are formed along zigzag-shaped elevations, in particular fold peaks, of a medical product that is formed in zigzag shape, in particular folded. In principle, the formation of barbs may be performed prior or subsequent to profiling of the medical product, in particular of its main surfaces.

Alternatively, the medical product is in the form of a hollow body, preferably in the form of a tube, wherein it is preferred that the ends of the tube or hose are open. The hollow body can also be an extrusion product, for example, an extrusion tube. One main surface of the medical product is preferably designed as the outer face, and the other main surface as the inner face, of the hollow body. In this form, the barbs generally protrude outwards.

In an advanced form, the medical product formed in a hollow body, in particular a hose or a tube, is surrounded by an overtube (sleeve), preferably for reinforcement, upon fitting the product with a surgical needle, for example. The overtube may be a textile fabric, in particular a mesh fabric, like a braiding (braided hose), for example. As an alternative, the overtube may be provided in the form of a grid (hose grid). By the overtube, the mechanical stability of the product may be improved in a particular beneficial manner. This applies in particular to passage of the medical product through biological tissue. A further contemplation is that the overtube will be removed again after implantation of the medical product into the body of a patient.

The biocompatible material of the medical product is generally a synthetic polymer. The polymer can be provided as a homopolymer, copolymer, terpolymer, tetrapolymer or the like. In particular, the biocompatible material can be provided as a block copolymer or block terpolymer. Polymer mixtures (polymer blends) are also possible.

The biocompatible material can in principle be a resorbable or non-resorbable or partially resorbable polymer. The resorbable polymer is preferably polylactide, polyglycolide, poly-ε-caprolactone, poly-p-dioxanone, polytrimethylene carbonate, polyhydroxybutyric acid, copolymers thereof, terpolymers thereof and mixtures thereof. The biocompatible material may be a copolymer or terpolymer, in particular a block copolymer or block terpolymer, comprising at least one monomer from the group including lactide, glycolide, trimethylene carbonate, paradioxanone, ε-caprolactone and hydroxybutyric acid. A suitable block terpolymer is based, for example, on glycolide, trimethylene carbonate and ε-caprolactone.

The non-resorbable polymers for the biocompatible material are preferably polymers from the group including polyolefins, polyesters, polyurethanes, polyamides, copolymers thereof terpolymers thereof and mixtures thereof. For example, the non-resorbable polymers can be polymers from the group including polypropylene, polyethylene terephthalate, polytetrafluoroethylene, nylon, silk, cotton, copolymers thereof, terpolymers thereof and mixtures thereof.

The biocompatible material may be a natural polymer, a so-called “biopolymer,” or a polymer derived therefrom. Preferred are proteins, in particular extracellular proteins, and/or polysaccharides and/or combinations thereof. Appropriate natural polymers or biopolymers may be selected from the group consisting of collagen, gelatin, elastin, reticulin, albumin, hyaluronic acid, starch, amylose, amylopectin, methyl cellulose, hydroxymethyl cellulose, hydroxyethyl cellulose, hydroxypropyl cellulose, hydroxyburyl cellulose, hydroxyethylmethyl cellulose, hydroxypropylmethyl cellulose, carboxymethyl cellulose, dextran, chondroitin sulfate, keratan sulfate, alginic acid, chitosan, heparin, salts thereof, derivatives thereof, and combinations thereof, for example.

Preferably, the medical product has an additivation. In other words, the product may include additives. Appropriate additives may be selected from the group consisting of biological agents, medical agents, pharmaceutical agents, cells, and other additives, like radiocontrast agents, and combinations thereof, for example. Preferably, the additives are selected from the group consisting of antimicrobial, in particular antibiotic agents, disinfecting agents, anti-inflammatory agents, growth-promoting agents, odor-controlling agents, analgetic agents, somatic cells, in particular fibroblasts and/or chondrocytes, precursor cells, in particular stem cells, barium sulfate, and combinations thereof. Particularly preferred is that the medical product has added differentiation factors, growth factors, recruiting factors and/or adhesion factors. Examples of growth factors may be fibroblast growth factor (FGF), transforming growth factor (TGF), platelet derived growth factor (PDGF), epidermal growth factor (EGF), granulocyte-macrophage colony stimulation factor (GMCSF), vascular endothelial growth factor (VEGF), insuline-like growth factors (IGF), hepatocyte growth factor (HGF), interleucin-1 B (IL-1 B), interleucin-8 (IL-8) and/or nerve growth factor (NGF). Particular emphasis is given to the option of adding cells, in particular somatic cells, generally autologous somatic cells, to the medical product. Depending on the field of application, the medical product may be provided with specific cell additions. For example, if the medical product is intended for use in a treatment of meniscus lesions and/or tendon lesions, an addition of chondrocytes may be advantageous. Another advantage is that substances produced and secreted by the cells may be an active aid in wound healing, wherein the cells themselves will synthesize and secret according active agents, in particular differentiation factors, growth factors, recruiting factors and/or adhesion factors. Furthermore, cellular produced substances may be beneficial with the application of the product in plastic surgery. Collagen formed in cells may enhance smoothing of wrinkles in plastic surgery, for example. All in all, a cellular additivation or colonization of the product is promoted by the barbs acting as surface enlarging structures.

As mentioned above, forming the medical product as a hose or tube (hose-type of tubular hollow body) is preferred. Particularly preferred is in this case, that the interior, if necessary exclusively the interior, of the hose or tube includes additives. As to the question of additives, reference is made to the entire above explanations. However, particular emphasis is given to addition of cells, differentiation factors, growth factors, cellular recruiting factors and/or cellular adhesion factors to the interior of the hose or tube. In that context, the interior of a medical product formed as a hose or tube may be completely or only partially filled with additives. In the case of cell additions, another option is to specifically inoculate the product interior with cells. In the case of a hose-type or tubular medical product, release of additives may be effected at target sites of the medical product, where the product material is perforated. The interior of a hose-type or tubular medical product may be at least partially, in particular completely filled with a resorbable material. Regarding a suitable resorbable material reference is made to the afore-described examples.

The product can also be present in sterilized form and, in particular, in a packaged form. In principle, the medical product can be sterilized using all sterilization methods known per se, particularly electron irradiation, γ-sterilization, X-radiation, ethylene oxide gas sterilization, steam sterilization and/or plasma sterilization.

The product can generally be used as an implant, in particular for fixing biological tissues, particularly human and/or animal tissues. The tissues to be fixed can be skin, fat, fascia, bones, muscles, organs, nerves, blood vessels, connective tissues, tendons or ligaments. The product is particularly suitable for use as a wound closure system or wound closure device, in particular as a self-fixing or knotless wound closure system or wound closure device. The medical product is also suitable for the apposition of biological tissues. The product is used preferably in plastic surgery, in particular for tightening the skin. In addition, the product is also suitable for use in minimally invasive surgery, particularly laparoscopic surgery. A further area of use concerns the fixing of implants, preferably hernia meshes, hernia plugs, prolapse meshes, urinary incontinence meshes, tissue scaffolds and tissue replacement materials, for example, collagen fleece, vein patches or neuro patches.

A further aspect concerns a surgical kit or set which comprises the medical product and at least one surgical needle or a combination of needle and thread. Provision can easily be made for the kit or set to comprise two surgical needles. For further details and features, particularly in respect of the medical product, reference is made to the preceding description.

This disclosure also relates to a method for producing the product, wherein a preliminary stage of the medical product, which is made of a biocompatible material and has two mutually opposite main surfaces, is cut to form barbs, with the material of the preliminary stage being broken through completely between the two main surfaces, and the barbs then being converted to a position in which they protrude relative to at least one of the two main surfaces.

Preferably, a planar, in particular strip-shaped or band-shaped preliminary stage is used for producing the medical product. Alternatively, a hollow body, particularly in the form of a tube, is used as the preliminary stage for producing the medical product. An extrusion product, in particular an extrusion film or an extrusion tube is preferably used as the preliminary stage. For further features and details in this connection, reference is made to the preceding description.

Preferably, the barbs are cut mechanically, preferably by at least one cutting blade. Conventional cutting devices, comprising a cutting bed, at least one cutting blade and holding or fixing elements, for example, vices, chucks, holding or clamping jaws, can be used. The barbs can also be produced by punching.

The barbs may be cut thermally, particularly with the aid of laser cutting methods. Lasers that can be used are, in principle, gas lasers, for example, CO₂ lasers, and also solid-state lasers, for example, Nd:YAG lasers. A suitable laser cutting machine generally comprises a laser beam source, a beam guide, and an at least movable focusing lens (concave mirror or positive lens). The beam leaving the beam source is either guided by fiber-optic cables, for example, in a Nd:YAG laser, or by a deflecting mirror, for example, in a CO₂ laser, to the machining lens which focuses the laser beam and in this way generates the power densities needed for the cutting, generally in the range of 10⁶ to 10⁹ watt/cm². Corresponding laser cutting methods are sufficiently known such that further details are not given here.

Preferably, additional barbs, called lateral barbs, are cut on at least one side surface, in particular two side surfaces, preferably two mutually opposite side surfaces. The lateral barbs are preferably cut into the preliminary stage of the medical product. However, it is also possible first to cut the preliminary stage of the medical product to form barbs on at least one of the two main surfaces, in particular both main surfaces, and thereafter to cut lateral barbs (two-stage method). Particularly preferably, additional barbs are cut at least on one longitudinal side surface, particularly two longitudinal side surfaces, preferably two mutually opposite longitudinal side surfaces, of the preliminary stage. In this way, as has already been mentioned, the number of barbs in total can be increased preferably in the longitudinal direction of the medical product. In addition to the cutting techniques that have already been mentioned, it is possible to cut lateral barbs using a cutting technique that is based on the use of a suitable cutting wire, in particular a metal wire. A heated cutting wire, in particular an electrically heated cutting wire, is preferably used. The cutting wire can in particular be a fine wire. A cutting wire with a diameter of between 20 and 50 μm is preferably used. Lateral barbs are preferably formed by laser cutting.

Suitably, the barbs are converted to the protruding position by drawing the medical product.

To convert the barbs to a position in which they protrude relative to at least one of the two main surfaces, an underpressure, in particular a vacuum, can be applied. The barbs may be converted to the protruding position by exposure to heat, particularly in the form of a stream of hot air. In principle, the barbs can also be converted mechanically from the plane of the product to the protruding position, particularly with the aid of a mechanical device that comprises fine pins, or with the aid of a stamp. For further details and features, particularly in respect of the angles at which the barbs can protrude relative to at least one of the two main surfaces, reference is likewise made to the preceding description.

The barbs can also be cut into a drawn or undrawn preliminary stage of the medical product. If the barbs are cut into an undrawn preliminary stage, a drawing procedure is then generally carried out. The drawing can be carried out in particular by heat, for example, in a temperature range of between 20 and 70° C. above the glass transition temperature of the material of the preliminary stage or of the medical product. Warm water or infrared radiation, for example, can serve to produce a heat that is advantageous for the drawing procedure. The drawing procedure can be carried out using a roller system in which the rollers can have different speeds of rotation. In particular, each subsequent roller can have a higher speed of rotation than the preceding roller of the drawing system. As an alternative to the continuous drawing just described, it is also possible to carry out intermittent drawing. For the intermittent drawing, the medical product can be clamped between the clamping jaws of a drawing device and then drawn.

The medical product may be subject to various post-treatment steps, in particular subsequent to completed drawing. To that end, the medical product may be subject to thermal treatment in a vacuum or low-pressure atmosphere, for example.

Further features will become clear from the following description of examples and the drawings. The individual features can be realized either singly or severally in combination. The drawings serve merely for illustration and better understanding and are not to be understood as in any way limiting the invention.

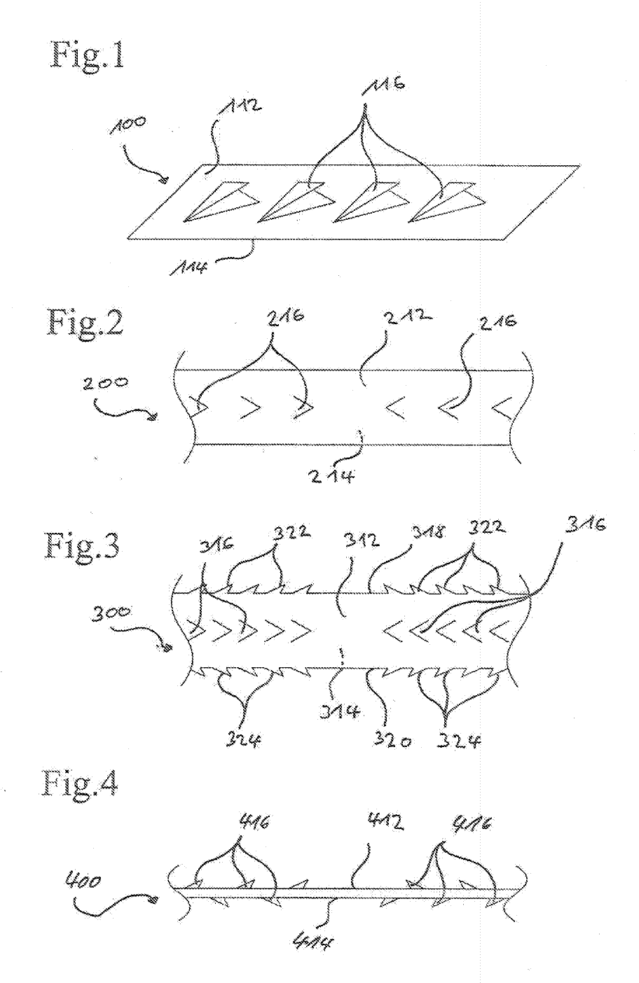

FIG. 1 shows a product 100. The product 100 has two main surfaces 112 and 114. On the main surface 112, V-shaped barbs 116 have been formed that break completely through the material of the product. The barbs 116 are unidirectional and are formed in a row arrangement on the main surface 112.

FIG. 2 shows a plan view of a product 200 with two main surfaces 212 and 214. On the main surface 212, the product 200 has a bidirectional arrangement of V-shaped barbs 216 that completely break through the material of the product. The barbs 216, in a first surface portion, are formed in the direction of the center of the product 200 and, in another, second surface portion, are likewise formed in the direction of the center of the product 200.

FIG. 3 shows a plan view of a product 300 with two main surfaces 312 and 314. On its main surface 312, the product 300 likewise has a bidirectional arrangement of barbs 316, in which the barbs 316, in a first surface portion, are formed in the direction of the center of the product 300 and, in another, second surface portion, are likewise formed in the direction of the center of the product 300. In addition, the product 300 has additional barbs 322 and 324, respectively, on its two longitudinal side surfaces 318 and 320. The barbs 322 and 324 are also in a bidirectional arrangement on the longitudinal side surfaces 318 and 320, wherein the barbs 322 and 324, in a first surface portion, are formed in the direction of the center of the longitudinal side surfaces 318, 320, respectively, and, in another, second surface portion, are likewise formed in the direction of the center of the longitudinal side surfaces 318, 320, respectively. The barbs 316 are likewise formed completely breaking through the material of the product, whereas the barbs 322 and 324 are formed by lateral cuts made in the material of the product.

FIG. 4 shows a schematic side view of a product 400. The product 400 has barbs 416 on both main surfaces 412 and 414. Relative to the main surfaces 412 and 414, the barbs 416 are each formed, in a first surface portion, in the direction of the center of the main surface 412, 414, respectively, and, in another, second surface portion, are likewise formed in the direction of the center of the main surface 412, 414, respectively. In addition, the barbs 416 of the main surfaces 412 and 414 are in each case offset relative to one another.

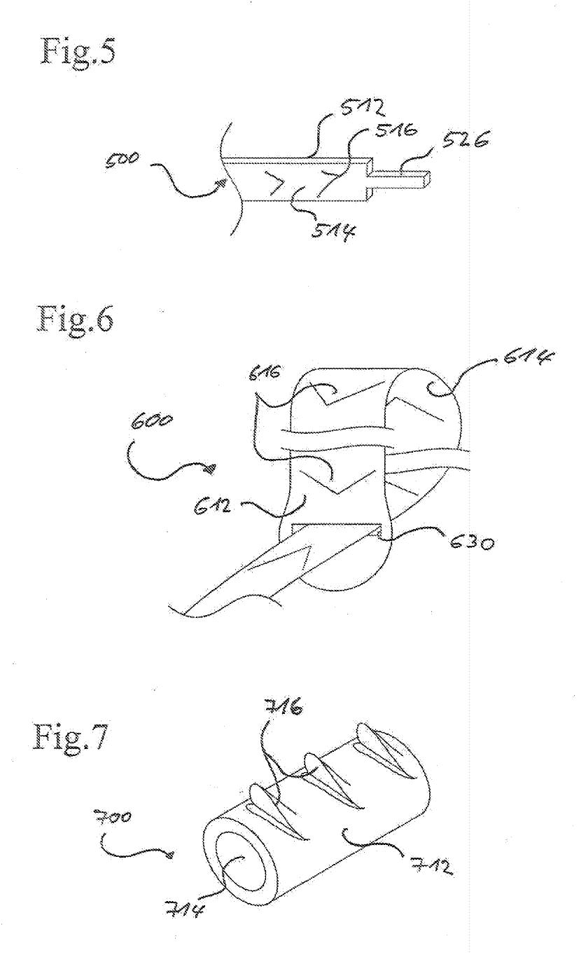

FIG. 5 shows a schematic view of a product 500 with two main surfaces 512 and 514 and with barbs 516, one end of the product having a web-shaped extension 526 for fitting with a surgical needle. For this purpose, the extension 526 is inserted through a hole in the area of the shaft end of a needle, and the needle is then crimped in the area of the hole. The extension 526 preferably has a width corresponding to the thickness of the product 500.

FIG. 6 shows a schematic view of a product 600 with two main surfaces 612 and 614 and with barbs 616, which product has a slit-shaped opening 630 at one of its ends. The opening 630 is formed in the transverse direction of the product 600. After a first passage through tissue, the product 600 can be guided through the opening 630 from its other end, in which case the barbs 616 advantageously prevent the product 600 from sliding back out of the product 600 through the opening (cable tie principle). This results in the formation of a knotless loop which constitutes a first point of fixation in a biological tissue.

FIG. 7 shows a schematic view of a product 700 in the form of a hollow body whose one main surface 712 is designed as the outer face, and the other main surface 714 as the inner face, of the hollow body 700. The main surface 712 has outwardly protruding barbs 716, which serve to anchor the hollow body 700 in biological tissues.

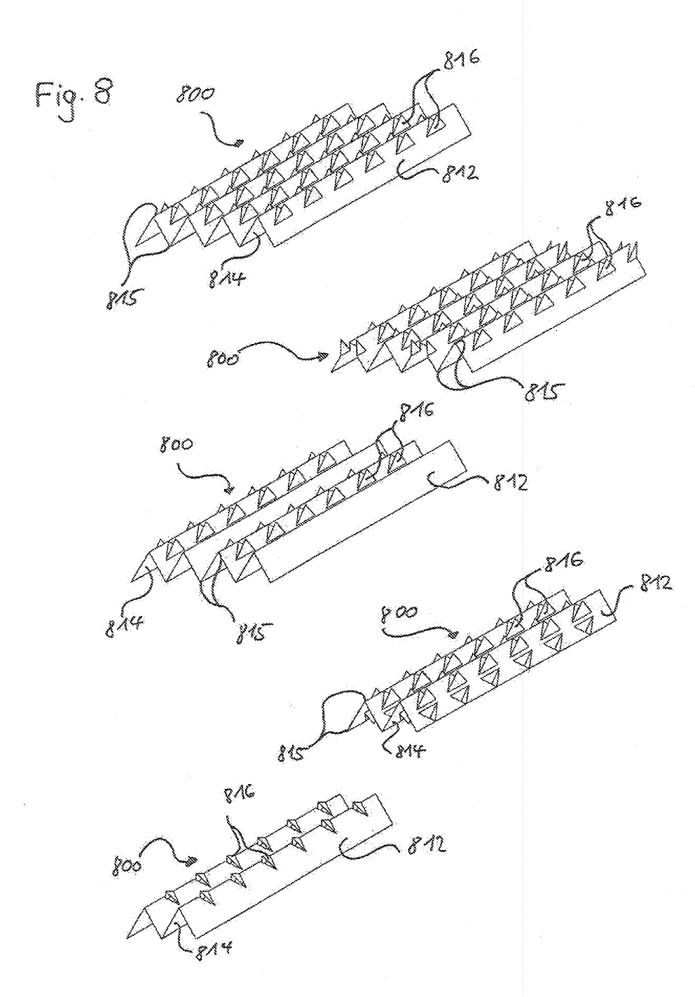

FIG. 8 is a schematic illustration of several variants of a product 800 formed in a zigzag or curved shape. Thus, the product 800 has two zigzag-shaped or curved main surfaces 812 and 814. Barbs 816 are formed along the zigzag-shaped or curved elevations 815. In principle, only the zigzag shaped elevations 815 of one main surface may comprise barbs (FIGS. 8a-c and 8e). Alternatively, all zigzag shaped elevations may comprise barbs 816 (FIG. 8d). In particular, the barbs 816 may have an offset arrangement. The formation of barbs at the zigzag-shaped or curved elevations 815 contributes to a firm and particularly secure anchoring of the medical product 800 to biological tissues, preferably of a patient.

### Example 1

### Extrusion of a Film Tape from PDO (poly-p-dioxanone)

Poly-p-dioxanone (PDO, inherent viscosity 1.61 dl/g at c=0.8 g/dl, 30° C., hexafluoro-2-propanol) was dried overnight and processed into the shape of a tape on a Haake TW100 twin-screw extruder at a spinning head temperature of 170° C., through a slit nozzle with a diameter of 1 mm and a width of 5 mm, and at a screw speed of 12 rpm. After leaving the spinning nozzle, the tape passed through an airgap of 3 cm and then a quenching bath (T=20° C.). The tape was taken off at a speed of 5 mm/min. The undrawn tape had a thickness of 0.6 mm and a. width of 4.1 mm. Thereafter, the tape was drawn. The drawing was carried out between two unheated septets, of which the second septet ran 4.5 times as fast as the first septet. After the drawing, the tape had a thickness of 0.30 mm and a width of 1.8 mm.

### Example 2

### Cutting Barbs into a PDO Film Tape

With the aid of a laser cutting device, barbs were Cut into a film tape prepared according to Example 1, specifically into a main surface of the tape, and completely breaking through the material of the film tape. In addition, barbs were also cut into both longitudinal surfaces. The mutual spacing of the lateral barbs was ca. 1 mm. The barbs on the main surface had a length of 1.0 mm.

### Example 3

### Preparation of a PDO Film Tape with Barbs Cut into it in the Undrawn State of the Film Tape

Poly-p-dioxanone (PDO, inherent viscosity 1.61 dl/g at c=0.8 g/dl, 30° C., hexafluoro-2-propanol) was dried overnight and processed into the shape of a tape on a Haake TW100 twin-screw extruder at a spinning head temperature of 170° C., through a slit nozzle with a diameter of 1 mm and a width of 5 mm, and at a screw speed of 12 rpm. After leaving the spinning nozzle, the tape passed through an airgap of 3 cm and then a quenching bath (T=20° C.). The tape was drawn off at a speed of 5 mm/min. The undrawn tape had a thickness of 0.6 mm and a width of 4.1 mm. Thereafter, barbs were cut into the undrawn tape with the aid of a laser cutting device. Barbs were cut into one of the two main surfaces of the tape, completely breaking through the material of the tape, and also into both longitudinal side surfaces of the tape. Thereafter, the incised tape was drawn intermittently in a water bath at a temperature of 48° C.

1. A medical product comprising a biocompatible material comprising two mutually opposite main surfaces, wherein at least one of the two main surfaces has barbs that anchor biological tissues, wherein the barbs protrude from the at least one main surface and are formed by cuts made in the biocompatible material, and wherein the material between the two main surfaces is completely broken through in an area of the cuts.

2. The medical product according to claim 1, wherein the barbs on the at least one main surface have a unidirectional or bidirectional arrangement.

3. The medical product according to claim 1, wherein the barbs for a first surface portion of the at least one main surface are formed in the direction of another, second surface portion of the at least one main surface and, for the other second surface portion, are formed in the direction of the first surface portion.

4. The medical product according to claim 1, wherein the barbs for a first surface portion of the at least one main surface are formed in the direction of the center of the at least one main surface and, for another, second surface portion of the at least one main surface, are formed in the direction of the center of the at least one main surface.

5. The medical product according to claim 1, wherein the barbs in the longitudinal direction of the at least one main surface have a row arrangement, an offset arrangement, a zigzag arrangement, a meandering arrangement, an arbitrary arrangement, or combinations of these.

6. The medical product according to claim 1, wherein the at least one main surface has at least two sets of barbs, and the sets of barbs are arranged parallel to one another on the at least one main.

7. The medical product according to claim 1, wherein the barbs are scale-shaped, escutcheon-shaped, shield-shaped, V-shaped, W-shaped, wedge-shaped, thorn-shaped or arrow-shaped.

8. The medical product according claim 1, wherein the barbs protrude from the at least one main surface at an angle α of between 0 and 90 degrees.

9. The medical product according to claim 1, wherein the barbs in the longitudinal direction of the medical product have a mutual spacing of 0.1 to 5 mm.

10. The medical product according to claim 1, wherein the barbs are formed on both main surfaces.

11. The medical product according to claim 1, wherein the barbs are offset relative to one another on mutually opposite main surfaces.

12. The medical product according to claim 1, wherein barbs are also formed on at least one side surface of the medical product.

13. The medical product according to claim 1, wherein the barbs are formed on at least one longitudinal side surface of the medical product.

14. The medical product according to claim 1, wherein the barbs are formed by cuts made in the medical product when the latter is in a drawn state.

15. The medical product according to claim 1, wherein the barbs are formed by cuts made in the medical product when the latter is in an undrawn state.

16. The medical product according to claim 1, wherei,. in areas without barbs, it has a square, in cross section.

17. The medical product according to claim 1, having a greater width than thickness.

18. The medical product according to claim 1, having a width-to-thickness ratio of between 100 and 1.

19. The medical product according to claim 1, wherein it is strip-shaped or band-shaped and designed as a band.

20. The medical product according to claim 1, in the form of an extrusion film.

21. The medical product according to claim 1, in the form of a film tape.

22. The medical product according to claim 1, wherein, at least at one end, it has web-shaped extension for fitting with a surgical needle, wherein the extension has a width corresponding to the thickness of the medical product.

23. The medical product according to claim 1, wherein, at one end, it has a slit-shaped opening provided to allow the medical product to be pulled through from its other end as a cable tie.

24. The medical product according to claim 1, in the form of a hollow tube or a hose, wherein one main surface is an outer face, and another main surface is an inner face of the hollow body.

25. The medical product according to claim 1, wherein the biocompatible material is a resorbable polymer selected from the group consisting of polylactide, polyglycolide, poly-ε-caprolactone, poly-p-dioxanone, poly trim ethylene carbonate, polyhydroxybutyric acid, copolymers thereof, terpolymers thereof and mixtures thereof.

26. The medical product according to claim 1, wherein the biocompatible material is a copolymer and/or terpolymer comprising at least one monomer selected from the group consisting of lactide, glycolide, trimethylene carbonate, para-dioxanone, ε-caprolactone and hydroxybutyric acid.

27. The medical product according to claim 1, wherein the biocompatible material is a non-resorbable polymer, selected from the group consisting of polyolefin, polyurethane, polyester, polyamide, copolymers thereof, terpolymers thereof and mixtures thereof.

28. The medical product according to claim 1, wherein the biocompatible material is polypropylene, polyethylene terephthalate, polytetrafluoroethylene, nylon, copolymers thereof, terpolymers thereof and mixtures thereof.

29. The medical product according to claim 1, for use as an implant, in particular for fixing biological tissues, particularly human and/or animal tissues.

30. The medical product according to claim 1, used as a self-fixing or knotless wound closure system.

31. The medical product according to claim 1, used in plastic surgery for tightening the skin.

32. The medical product according to claim 1, used as a fixing means for implants in biological tissues.

33. A surgical kit comprising a medical product according to claim 1 and at least one surgical needle.

34. A method for producing a medical product according to claim 1, wherein a preliminary stage of the medical product, which is made of a biocompatible material and has two mutually opposite main surfaces, is cut to form barbs, with the material of the preliminary stage being broken through completely between the two main surfaces, and the barbs are then converted to a position in which they protrude relative to at least one of the two main surfaces.

35. The method according to claim 34, wherein a planar, strip-shaped or band-shaped preliminary stage produces the medical product.

36. The method according to claim 34, wherein a hollow body of a tube or a hose is the preliminary stage for producing the medical product.

37. The method according to claim 34, wherein an extrusion film, an extrusion tube or an extrusion hose, is used as the preliminary stage for producing the medical product.

38. The method according to claim 34, wherein the barbs are cut mechanically by at least one cutting blade and/or by punching.

39. The method according to claim 34, wherein the barbs are cut thermally using laser cutting techniques.

40. The method according to claim 34, characterized in that additional barbs are cut on at least two side surfaces of the preliminary stage.

41. The method according to claim 34, wherein the barbs are converted to the protruding position by drawing the product.

42. The method according to claim 34, wherein the barbs are converted to the protruding position by application of an underpressure.

43. The method according to claim 34, wherein the barbs are converted to the protruding position by being exposed to heat in the form of a stream of hot air.

44. The method according to claim 34, wherein the barbs are converted to the protruding position mechanically with the aid of a stamp.
